(19) United States
(12) Patent Application Publication
(10) Pub. No.: US 2008/0020439 A1
(43) Pub. Date: Jan. 24, 2008

(54) Enzymes As Corrosion Inhibitors By Removal Of Oxygen Dissolved In Water

(75) Inventors: Mattia De Dominicis, Montvale, NJ (US); Liliana Oliva, Summaga di Portogruaro (Ve) (IT)

Correspondence Address: NORRIS, MCLAUGHLIN & MARCUS, 875 THIRD AVE, 18TH FLOOR, NEW YORK, NY, 10022, US

(73) Assignee: RECKITT BENCKISER N.V., Hoofddorp (NL)

(21) Appl. No.: 10/598,435

(22) PCT Filed: Mar. 2, 2005
(86) PCT No.: PCT/GB05/00813

§ 371 Date: Nov. 7, 2006

(30) Foreign Application Priority Data
Mar. 2, 2004 (GB) 0404658.7

(51) Int. Cl. C09K 3/00; B01D 53/84; C12N 9/02

(52) U.S. Cl. 435/189; 435/266

(57) ABSTRACT

The invention relates to a new process for water de-oxygenation, for application in aerosol products. The process involves the use of an enzymatic system based on an oxidase enzyme a substrate for the oxidase enzyme and catalase. These two enzymes consume oxygen by a two step reaction with the substrate and hydrogen peroxide, which is formed in the first reaction.

The invention relates to a new process for water de-oxygenation, for application in aerosol products. The process involves the use of an enzymatic system based on an oxidase enzyme a substrate for the oxidase enzyme and catalase. These two enzymes consume oxygen by a two step reaction with the substrate and hydrogen peroxide, which is formed in the first reaction.

Corrosion reactions take place in the presence of oxygen, oxygen dissolved in water is responsible for can corrosion in aqueous based formulations within aerosol containers. Currently a number of techniques are used to minimise corrosion in aerosol cans, for example, by the use of corrosion inhibitors or by an oxygen displacement process using nitrogen gas. We have found that corrosion is severely retarded if oxygen is substantially removed from the water present in the aerosol can.

Examples of products found in aerosol cans are air care products, household products, fabric care, waxes, polishes, insecticides, ironing aids, fabric refreshers and carpet cleaners.

The aerosol canister is metal, preferably steel or tin coated steel.

The world market trend is to move towards aerosol formulations containing more water. This is due mainly to regulatory issues: the reduction of the volatile organic content (VOC) level in aerosol products has involved a reduction in the amount of solvent of many products and an increase in the water content.

When aerosol compositions contain less than 50 ppm of water, corrosion of the aerosol can is not generally a serious problem. However, if the water content is more than 50 ppm in the aerosol composition then corrosion is more likely to occur.

Many corrosion inhibitor systems have been developed for facing these new regulatory requirements. Examples of these products are borates, benzoates, molybdate, special surfactants (such as sodium lauroyl sarcosinate), sodium nitrite and morpholine and silicates. Usually an acceptable control of the corrosion during the product life of the aerosol canister (around 2 years) is built in to the composition. The above corrosion inhibitors tend to interact with the aerosol canister's surface providing protection against corrosion.

There also can be negative effects of corrosive de-tinning on the performance of the product. The yellow tin corrosion complex may remain especially when sprayed onto white surfaces. White fabrics or carpets can remain coloured by the liquids of aged aerosol products. Other considerations relate to certain stains like coffee, tea and wine that contain cationic metals. These metals can form brown coloured complexes with tin hydroxyl, causing an evident negative effect of the cleaning formulation onto overall cleaning performance.

Therefore, there is a need to identify better ways to prevent corrosion in aerosol canisters.

Corrosion is an electrochemical process. All corrosion reactions are started by the presence of water and oxygen. Oxygen is a direct participant in the corrosion reaction, acting as a cathode-accepting electron.

Dissolved oxygen present in water based formulations within aerosols is one of the most important factors influencing the rate of corrosion for all metals.

Many corrosion inhibitors have been identified in the prior art, but none really halt dissolution of the tin layer in tin-plated aerosol cans over the two years standard can life, they merely slow it down. Even resin lacquered tin-plated cans generally need an effective corrosion inhibitor system.

T. Godfrey, J. Reichelt: Industrial Enzymology, Nature Press 1983—Chapter 4.2: G.Richter—Glucose Oxidase, U.S. Pat. No. 5,980,956, EP 0818960 & EP 0835299 describe the use in the food industry and especially in canned soft drinks industry of an enzymatic system based on glucose oxidase and catalase as an antioxidant primarily to prevent changes in colour and flavour of foods products both during processing and in storage.

U.S. Pat. No. 4,414,334 describes the use of alcohol oxidase and catalase to remove oxygen dissolved in aqueous liquids and discloses the use of such systems in foodstuffs and water distribution systems.

Currently a vacuum process is used to remove oxygen during aerosol product manufacture, which does reduce the oxygen content in the aerosol can. The reduction is only in the aerosol can head space and has little effect on the deoxygenation of the liquid phase. For liquid phase deoxygenation currently used is a method called nitrogen stripping, a process that is quite expensive. The process of the present invention can reduce the oxygen content in the aerosol can during manufacturing and even, during product storage.

We have found that the use of an oxidase enzyme and a substrate for the oxidase enzyme combined with catalase effectively reduces the rate of corrosion in aerosol cans by reducing almost to zero the concentration of oxygen dissolved in the water.

The process of the invention is particularly effective at neutral and acidic pH. The deoxygenating process requires a longer time at alkaline pH: this is not necessarily a problem since the enzymatic system will continue to work over time if placed in the aerosol product.

Other advantages of enzymes are that they are very effective even at low concentration, starting from 0.01 ppm of enzyme and 50 ppm of substrate. The enzymes are also compatible with aerosol formulations and have a low impact on the overall formulation cost.

We present as a feature of the invention an aerosol product comprising a sealed metal canister containing an aerosol composition comprising an oxidase enzyme and a substrate for the enzyme. Preferably catalase is also added.

Alternatively we present as a feature of the invention a method of deoxygenating an aerosol produce comprising filling an aerosol canister with an aerosol composition, an oxidase enzyme and a substrate for the oxidase enzyme and, in any order, filling the aerosol canister with propellant and sealing the aerosol canister.

Preferably a catalase is additionally added into the canister.

Alternatively, we present as a feature of the invention use of an oxidase enzyme and a substrate for the oxidase enzyme as a corrosion inhibiting system for aerosol products. Preferably catalase is also used.

Suitable oxidase enzymes are those classified under enzyme classification E.C.1.1.3 (Acting on the CH—OH group of donors with oxygen as acceptor) and include one or more of the following. Not all enzymes produce hydrogen peroxide as a product of the reaction. Therefore in a preferred feature of the invention when such enzymes are used the presence of catalase is not required, for example nucleoside oxidase.

Preferred enzymes are selected from one or more of the following; Malate oxidase, Glucose oxidase, Hexose oxidase, Cholesterol oxidase, Aryl-alcohol oxidase, L-gulonolactone oxidase, Galactose oxidase, Pyranose oxidase, L-sorbose oxidase, Pyridoxine 4-oxidase, Alcohol oxidase, Catechol oxidase, (S)-2-hydroxy-acid oxidase, Ecdysone oxidase, Choline oxidase, Secondary-alcohol oxidase, 4-hydroxymandelate oxidase, Long-chain-alcohol oxidase, Glycerol-3-phosphate oxidase, Xanthine oxidase, Thiamine oxidase, L-galactonolactone oxidase, Cellobiose oxidase, Hydroxyphytanate oxidas, Nucleoside oxidase, N-acylhexosamine oxidase, Polyvinyl-alcohol oxidase, Methanol oxidase, D-arabinono-1,4-lactone oxidase, Vanillyl-alcohol oxidase, Nucleoside oxidase, D-mannitol oxidase and Xylitol oxidase.

A preferred enzyme is Glucose Oxidase. Glucose Oxidase is a highly specific enzyme derived from the fungi Aspergillus Niger and Penicillinum. Glucose oxidase is an oxidoreductase, that catalyses the oxidation of D-Glucose to gluconic acid using molecular oxygen and releasing hydrogen peroxide. Glucose oxidase has a molecular weight of 192000, an optimium temperature of 30-50° C. and optimum pH of 4.5-6.5. It is inhibited by heavy metal salts, preferably a chelating agent may be added to the aerosol composition, and sulfhydyl chelating agents. The effective amount enzyme needed is from 0.001 ppm to 500 ppm, more preferably between 0.01 and 50 ppm.

Catalase is a common enzyme present in the cell of plants, animals and aerobic bacteria. It promotes the conversion of hydrogen peroxide to water and molecular oxygen. This reaction is very specific and very fast: catalase has one of the highest turnover rates for all enzymes. Catalase is inhibited by urea, freezing and sunlight under aerobic conditions. The effective amount of enzyme needed is from 0.001 ppm to 500 ppm, more preferably between 0.01 and 50 ppm.

The reaction is:
1. 2C₆H₁₂O₆+2H₂O+2O₂→2C₆H₁₂O₇+2H₂O₂
(glucose oxidase)
2. 2H₂O₂→2H₂O+O₂
(catalase)
Total reaction:
2C₆H₁₂O₆+O₂→2C₆H₁₂O₇

By forcing the equilibrium of the reaction by an excess of substrate to the oxidase enzyme, it is possible to end up with a final oxygen content close to zero.

Therefore, the concentration of substrate needed in order to increase the velocity of the first reaction is ideally greater than the Km of the enzyme selected (Km is the Michael's constant and is the affinity of the enzyme for the substrate, i.e. the concentration at which 50% of the enzyme binding sites are occupied). Typical Km's are 10⁻¹ M to 10⁻⁶ M.

An important feature of the invention is a substrate for the oxidase enzyme used, this may already be present in the composition to be packaged in the aerosol canister or it might be added. A preferred substrate is D-glucose.

The performance of the new corrosion inhibitor system has been evaluated first by measuring the dissolved oxygen reduction (Oxy-meter) in a typical conditions and then by a quick method for the evaluation of corrosion, the jar method, using as fill formulation using tap water treated or not with the enzymatic system on a standard epoxy coated piece of aerosol can.

Oxy-Meter Evaluation

A 5 L glass beaker is used in this test.
4 L of tap water are added into the beaker and warmed to 40° C.
pH of the solution is measured and adjusted to desired value. Dissolved oxygen (DO mg/L) pH and Temperature (° C.) are measured through an Oxy-Meter YSI 556 MPS.

The time zero DO value is collected, D-Glucose is added to the solution and immediately after the enzymatic system is dosed.

The reaction is then followed constantly reading the DO value until it reaches a plateau value.

The system is open, so no control to oxygen intake from the air is considered.

Jar Method:

50 ml glass jars with screw plugs are used in this test.

A round piece of a can is cut and applied on the internal surface of the jar screw plug. A cross is cut by a blade on the can piece in order to simulate possible defects on the can walls.
A poly tetra fluoroethylene gasket is also applied on the plug in order to guarantee a good sealing system. The jar is filled with the testing formula and it is stored in the inverted position to obtain the contact between the liquid formula and the tin plated can piece applied on the plug.

The storage is carried out at different temperature (20° C., 40° C. and 50° C.) for several days up to 1 month. The storage situation is monitored after 1 day, 1 week, 2 weeks, 1 months and compared to reference can pieces and liquids. The can piece appearance is recorded. A recording data table with the corresponding corrosion rating is reported below:

| Corrosion Rating JM | Can piece appearance |
|---|---|
| 0 | No difference from reference |
| 1 | Low darkening along the cut lines |
| 2 | Darkening along the cut lines |
| 3 | Strong darkening on all the can piece area |
| 4 | Darkening on all the can piece area |
| 5 | Evident darkening on all the can piece area |
| 6 | Rust |

### EXAMPLES

The liquid phases are typically prepared by mixing D-Glucose anhydrous to warm 40° C. tap water, adjusting the pH to the desired value and then adding the enzymatic system to start the de-oxygenation reaction.

TABLE 1

| | Ref 1 | Ref 2 | Ref 4 | Ref 5 | Ref 6 | Ref 7 |
|---|---|---|---|---|---|---|
| Components | ppm | ppm | ppm | ppm | ppm | ppm |
| D-Glucose | 60 | 250 | 500 | 1000 | 1000 | 500 |
| | (0.006%) | (0.025%) | (0.05%) | (0.05%) | (0.1%) | (0.05%) |
| OxyGo 1500 | 0.0125 | 0.05 | 0.1 | 0.2 | 0.2 | 0.1 |
| Tap Water | to 100% | to 100% | to 100% | to 100% | to 100% | to 100% |
| PH | 7 | 7.8 | 7.1 | 7.1 | 9.1 | 4.9 |

TABLE 2

| Component | Description of component |
|---|---|
| D-Glucose | D(+)-Glucose anhydrous >99.5% from Fluka |
| OxyGo 1500 | Glucose Oxidase Enzyme with Catalase side |
| | activity from Genencor |
| NaOH | Sodium Hydroxide, 10% solution |
| H2SO4 | Sulphuric Acid, 9% solution |

Example Results

The enzymatic corrosion inhibitor system was tested for all formulations using an Oxy-meter evaluation and for formulation Ref. 4, Ref. 5, Ref. 6 and Ref. 7 using the Jar method. Evaluation of possible residual H2O2, due to slow action of catalase, was done for formulation Ref. 4, Ref. 5, Ref. 6 and Ref. 7.

Results:

DO (mg/L) at 40° C.
(Oxy-meter evaluation)

| Product | Time 0 | 30′ | 60′ | 90′ |
|---|---|---|---|---|
| Ref 1 | 4.40 | 4.03 | 3.79 | 3.69 |
| Ref 2 | 4.24 | 4.12 | 3.42 | 3.18 |
| Ref 4 | 4.31 | 2.06 | 1.40 | 1.10 |
| Ref 5 | 4.51 | 1.05 | 0.84 | 0.60 |
| Ref 6 | 4.50 | 2.76 | 2.01 | 1.49 |
| Ref 7 | 3.80 | 1.55 | 1.48 | 1.25 |

| | Corrosion rating | | | | | | | |
|---|---|---|---|---|---|---|---|---|
| | (Jar method) | | | | | | | |
| | 1 day | | 1 week | | 2 weeks | | 1 month | |
| Product | uncut | cut | uncut | cut | uncut | cut | uncut | cut |

20° C.

| | | | | | | | | |
|---|---|---|---|---|---|---|---|---|
| Ref 4 | 0 | 0 | 0 | 0 | 0 | 0 | 0 | 1 |
| Ref 5 | 0 | 0 | 0 | 0 | 0 | 0 | 0 | 1 |
| Ref 6 | 0 | 0 | 0 | 0 | 0 | 1 | 0 | 1 |
| Ref 7 | 0 | 0 | 0 | 0 | / | / | / | / |
| Tap Water | 0 | 6 | 0 | 6 | 0 | 6 | 1 | 6 |

40° C.

| | | | | | | | | |
|---|---|---|---|---|---|---|---|---|
| Ref 4 | 0 | 0 | 0 | 0 | 0 | 1 | 0 | 1 |
| Ref 5 | 0 | 0 | 0 | 0 | 0 | 1 | 0 | 1 |
| Ref 6 | 0 | 0 | 0 | 0 | 0 | 1 | 0 | 1 |
| Ref 7 | 0 | 0 | 0 | 0 | / | / | / | / |
| Tap Water | 0 | 6 | 0 | 6 | 0 | 6 | 2 | 6 |

50° C.

| | | | | | | | | |
|---|---|---|---|---|---|---|---|---|
| Ref 4 | 0 | 0 | 0 | 0 | 0 | 0 | 0 | 1 |
| Ref 5 | 0 | 0 | 0 | 0 | 0 | 1 | 0 | 2 |
| Ref 6 | 0 | 0 | 0 | 0 | 0 | 2 | 0 | 2 |
| Ref 7 | 0 | 0 | 0 | 0 | / | / | / | / |
| Tap Water | 0 | 6 | 0 | 6 | 0 | 6 | 2 | 6 |

| | H2O2% formation | | | |
|---|---|---|---|---|
| Product | 10′ | 30′ | 60′ | 90′ |
| Ref 4 | 0.01 | 0.00 | 0.00 | 0.00 |
| Ref 5 | 0.00 | 0.00 | 0.00 | 0.00 |
| Ref 6 | 0.03 | 0.00 | 0.00 | 0.00 |
| Ref 7 | 0.01 | 0.00 | 0.00 | 0.00 |

The above results show that the two enzymatic reactions take place relatively quickly, so the H₂O₂ formed in the first step is consumed in the second step.

1. An aerosol product comprising a sealed metal canister containing an aerosol composition comprising an oxidase enzyme and a substrate for said oxidase enzyme.

2. An aerosol product according to in claim 1 wherein the aerosol composition additionally comprises catalase.

3. An aerosol product according to claim 1 wherein the aerosol composition comprises >50 ppm of water.

4. An aerosol product according to claim 1 wherein the oxidase enzyme is glucose oxidase and the substrate is D-glucose.

5. A method of deoxygenating an aerosol product comprising the step of:
supplying to an aerosol canister an oxidase enzyme and a substrate for the oxidase enzyme, an aerosol composition, and a propellant and thereafter, sealing the aerosol canister.

6. A method according to claim 5 wherein the method further includes supplying a catalase to the aerosol canister.

7. A method of inhibiting corrosion of a sealed and pressurized aerosol canister containing which method comprises the step of:
providing an aerosol composition comprising an oxidase enzyme and a substrate for the oxidase enzyme as a corrosion inhibiting system to the said aerosol canister.

8. A method according to claim 7 wherein the aerosol composition additionally comprises a catalase.

9. An aerosol product according to claim 2 wherein the aerosol composition comprises >50 ppm of water.

10. An aerosol product according to claim 2 wherein the oxidase enzyme is glucose oxidase and the substrate is D-glucose.
